(19) United States
(12) Patent Application Publication
(10) Pub. No.: US 2021/0324045 A1
(43) Pub. Date: Oct. 21, 2021

(54) BULLFROG SKIN-DERIVED COLLAGEN, MATERIALS COMPRISING THEREOF, AND APPLICATIONS IN WOUND HEALING

(71) Applicant: Nanyang Technological University, Singapore (SG)

(72) Inventors: Chor Yong TAY, Singapore (SG); Cigdem CIMENOGLU, Singapore (SG); Jun Kit WANG, Singapore (SG); Nguan Soon TAN, Singapore (SG); Swee Neo Cleo CHOONG, Singapore (SG)

(21) Appl. No.: 17/233,022

(22) Filed: Apr. 16, 2021

(30) Foreign Application Priority Data
Apr. 16, 2020 (SG) 10202003504W

(51) Int. Cl. C07K 14/78; A61L 15/32; A61L 26/00; A61L 15/42; C07K 1/36; A61L 15/44

(52) U.S. Cl. CPC C07K 14/78; A61L 15/325; A61L 26/0033; A61L 15/425; C07K 1/36; A61L 2400/12; A61L 2300/404; A61L 2300/236; A61L 2300/104; A61L 2300/406; A61L 15/44

(57) ABSTRACT

Disclosed herein is a polymeric material that includes a crosslinked polymer matrix formed from a non-mammalian collagen and a crosslinking agent and/or a crosslinked polymer matrix formed from a non-mammalian collagen that has undergone self-crosslinking, wherein the non-mammalian collagen is type I collagen. Also disclosed herein is a wound dressing that incorporates said material and methods of treatment of a wound with said polymeric material or said wound dressing.

### CROSS-REFERENCE TO RELATED APPLICATIONS

This application claims priority to Singapore Application No. SG 10202003504W, filed Apr. 16, 2020, which is herein incorporated by reference.

### BACKGROUND OF THE DISCLOSURE

### Field of Invention

This invention relates to a form of marine collagen that can be used in wound healing. As such, the invention relates to the use of said material in wound healing, as well as the manufacture of said material and wound dressings including said material.

### Description of the Related Art

The listing or discussion of a prior-published document in this specification should not necessarily be taken as an acknowledgement that the document is part of the state of the art or is common general knowledge.

Wound healing is a highly complex, dynamic biological and chemical process that involves step-wise cellular response to restore the anatomical function of impaired living tissue. There are 3 main stages in wound healing: inflammation, proliferation and remodelling. There are more than 300 million cases every year involving one or more of acute, traumatic and burn wounds. Thus, the number of patients who require medical intervention in order to solve issues relating to wound healing is significant. The complete wound healing process depends greatly on successful wound care and the global market for products associated with wound care is foreseen to be worth $15-22 billion by 2024 (C. K. Sen, Adv. Wound Care (New Rochelle) 2019, 8, 39-48). Wound dressings are the predominant type of wound care product, and there are more than 3000 different types of wound dressing fabricated from synthetic and natural polymers.

Among the different types of wound dressing, collagen is the most frequently-used natural polymer due to its superior biological and chemical properties. However, problems associated with the application of collagen in wound healing remain. These problems include collagen's rapid degradation rate, poor adhesion, and the slow proliferation and migration of cells (S. Chattopadhyay et al., Biopolymers 2014, 101, 821-833). Moreover, the majority of the collagen-based biomaterials are primarily fabricated from mammalian sources, such as bovine and porcine sources. This may lead to cultural and religious issues, as well as concerns about transmittance of disease. In addition, the extraction of collagen typically involves a complex, laborious, time-consuming, and expensive extraction process, due to the stiff and fibrous nature of bovine and porcine tissues. Thus, in recent years, researchers have shown an increasing interest in non-mammalian sources of collagen, such as aquatic collagen or jellyfish collagen. However, the applicability of marine collagen to wound healing has been stifled, due to their relatively poor thermal stability, which is associated with rapid denaturation and degradation of collagen from marine sources.

Type I collagen has been widely used as a wound dressing material due to its excellent bioactive properties such as biocompatibility, biodegradability, and cell-material interactions. Hence, most of the commercially available wound dressings are made of Type I collagen, such as Apligraf (Organogenesis Inc, Canton, Mass.), Dermagraft (Advanced Biohealing Inc, Westport, Conn.), BIOPAD™ (Angelini Pharma Inc.), Helix3® Bioactive Collagen (Amerx Health Care Corp.) and Stimulen™ Collagen Powder (Southwest Technologies, Inc.). However, most of these wound dressings are fabricated using mammalian-derived collagen.

Therefore, there remains a need for a collagen with improved properties that avoids one or more of the problems identified above.

### SUMMARY OF INVENTION

It has been surprisingly found that it is possible to modify marine collagen to enhance its structural and functional properties, and to develop cheaper and more efficient methods for “waste-to-resource” production of marine collagen-based wound healing materials. Aspects and embodiments of the current invention will be described by reference to the following numbered clauses.

1. A polymeric material comprising one or both of a crosslinked polymer matrix formed from a non-mammalian collagen and a crosslinking agent and a crosslinked polymer matrix formed from a non-mammalian collagen that has undergone self-crosslinking, wherein the non-mammalian collagen is type I collagen.

2. The polymeric material according to Clause 1, wherein the crosslinking agent is a pharmaceutically acceptable crosslinking agent.

3. The polymeric material according to Clause 1 or Clause 2, wherein the crosslinking agent is selected from one or more of the group consisting of genipin and compounds comprising two or more crosslinkable functional groups selected from the group consisting of amino, carboxylic acid, ester, aldehyde and epoxide functional groups (e.g. the crosslinking agent is selected from compounds comprising two or more crosslinkable functional groups selected from the group consisting of aldehyde and epoxide functional groups).

4. The polymeric material according to any one of the preceding clauses, wherein the crosslinking agent is selected from compounds comprising two crosslinkable functional groups.

5. The polymeric material according to Clause 3 or Clause 4, wherein the crosslinking agent is selected from one or more of the group consisting of glutaraldehyde and 1,4-butanediol diglycidyl ether (e.g. the crosslinking agent is 1,4-butanediol diglycidyl ether).

6. The polymeric material according to any one of the preceding clauses, wherein, when present, the crosslinking agent forms from 3 to 15 wt %, such as from 4 to 10 wt % of the crosslinked polymer matrix formed from a non-mammalian collagen and a crosslinking agent, optionally wherein, when present, the crosslinking agent forms about 5 wt % of the crosslinked polymer matrix formed from a non-mammalian collagen and a crosslinking agent.

7. The polymeric material according to any one of the preceding clauses, wherein, when the crosslinked polymer matrix is formed from a non-mammalian collagen that has undergone self-crosslinking, the non-mammalian collagen has been crosslinked by a transglutaminase.

8. The polymeric material according to any one of the preceding clauses, wherein the polymeric material further comprises an antibacterial compound, optionally wherein the antibacterial compound is selected from one or more of the group consisting of chitosan, silver nanoparticles and antibiotics (e.g. the antibacterial compound is silver nanoparticles).

9. The polymeric material according to any one of the preceding clauses, wherein the non-mammalian collagen is derived from a non-mammalian vertebrate.

10. The polymeric material according to any one of the preceding clauses, wherein the non-mammalian collagen is derived from bullfrog skin, optionally wherein the bullfrogs belong to the genus rana (e.g. the bullfrogs are of the species Rana catesbeiana).

11. The polymeric material according to any one of the preceding clauses, wherein the polymeric material is provided as a film, a sponge, a patch or a filling material.

12. The polymeric material according to Clause 11, wherein the polymeric material is provided as a sponge or a patch.

13. The polymeric material according to Clause 11 or Clause 12, wherein the sponge, the patch and the filling material are porous.

14. A wound dressing comprising a polymeric material as described in any one of Clauses 1 to 13.

15. A method of wound healing comprising the step of providing a suitable amount of a polymeric material according to any one of Clauses 1 to 13 to a subject in need thereof.

16. A method of wound healing comprising the step of providing a suitable amount of a wound dressing according to Clause 14 to a subject in need thereof.

17. A method of providing a collagen precursor mixture from a non-mammalian source, the method comprising the steps of:

(a) providing a mixture of pre-treated skins from a non-mammalian animal in an acidic solvent; and
(b) subjecting the mixture to mechanical blending to provide the collagen precursor mixture in the form of a paste.

18. The method according to Clause 17, wherein one or more of the following apply:

(ci) the acidic solvent is aqueous acetic acid, optionally wherein the aqueous acetic acid has a molarity of from 0.1 to 1 M, such as from 0.3 to 0.7 M, such as about 0.5 M;
(cii) the acidic solvent is provided in a weight to volume ratio of from 0.1:10 to 2:10, such as 1:10, where the weight refers to the weight of the pre-treated skins from a non-mammalian animal and the volume refers to the volume of the acidic solvent;
(ciii) the blending is conducted over a period of from 1 to 20 minutes, such as from 2 to 10 minutes, such as about 5 minutes; and
(civ) the blending is conducted at from 20,000 to 50,000 rpm, such as from 30,000 to 40,000 rpm, such as about 35,000 rpm;
(cv) the entire method is conducted at a temperature of from 0.1 to 10° C., such as from 1 to 5° C., such as about 4° C.;
(cvi) the pre-treated skins are bullfrog skin, optionally wherein the bullfrogs belong to the genus rana (e.g. the bullfrogs are of the species Rana catesbeiana).

19. A method of providing collagen from a non-mammalian source, the method comprising the steps of:

(aa) providing a collagen precursor mixture in the form of a paste;
(ab) diluting the paste with water and centrifuging the resulting diluted paste to provide a collagen solution and a pellet comprising pigments and collecting the collagen solution;
(ac) adding an inorganic salt to the collagen solution for a period of time (e.g. from 12 to 48 hours, such as from 18 to 24 hours) to precipitate out a collagen salt, which is then collected by centrifugation;
(ad) adding an acidic solvent to the collected collagen salt to provide a free collagen mixture and subjecting the free collagen mixture to dialysis to provide a solution of collagen from a non-mammalian source.

20. The method according to Clause 19, wherein one or more of the following apply:

(ba) the collagen precursor mixture is obtained using the method according to Clause 17;
(bb) the paste is diluted by water in a ratio of from 1:2 to 1:10 vol/vol, such as from 1:3 to 1:7 vol/vol, such as about 1:5 vol/vol or the paste is diluted by water in a ratio of from 1:10 to 1:30 vol/vol, such as from 1:10 to 1:20 vol/vol, such as about 1:10 vol/vol;
(bc) the centrifugation in step (ab) of Clause 19 is conducted at from 15,000 to 50,000×g, such as from 20,000 to 35,000×g, such as about 25,000×g;
(bd) the centrifugation in step (ab) of Clause 19 is conducted for a period of from 5 to 45 minutes, such as from 10 to 30 minutes, such as about 15 minutes;
(be) the inorganic salt in step (ac) of Clause 19 is selected from one or more of sodium sulphate, ammonium sulphate, potassium chloride and sodium chloride (e.g. the inorganic salt is sodium chloride), optionally wherein the inorganic salt is provided as an aqueous solution having a concentration of from 0.5 to 4.0 M, such as from 0.5 to 1.5 M, such as about 0.9 M;
(bf) the centrifugation in step (ac) of Clause 19 is conducted at from 3,000 to 10,000×g, such as from 4,000 to 6,000×g, such as about 5,500×g;
(bg) the centrifugation in step (ac) of Clause 19 is conducted for a period of from 5 to 45 minutes, such as from 10 to 30 minutes, such as about 15 minutes;
(bh) the acidic solvent in step (ad) of Clause 19 is aqueous acetic acid, optionally wherein the aqueous acetic acid has a molarity of from 0.1 to 1 M, such as from 0.3 to 0.7 M, such as about 0.5 M;
(bi) the dialysis in step (ad) of Clause 19 is conducted in two rounds, wherein:
(i) the first round dialysis makes use of aqueous acetic acid at a concentration of from 0.01 to 0.3 M, such as from 0.05 to 0.2 M, such as about 0.1 M; and
(ii) the second round dialysis makes use of water;
   (bj) the entire method is conducted at a temperature of from 0.1 to 10° C., such as from 1 to 5° C., such as about 4° C.;
   (bk) step (ac) of Clause 19 is only conducted once.

The method according to Clause 19 or Clause 20, wherein the solution of collagen from a non-mammalian source is lyophilised.

### BRIEF DESCRIPTION OF THE DRAWINGS

FIG. 1 depicts (a) schematic diagram of the extraction process employed to recover collagen from bullfrog skins; and (b) cross-linking reaction between acid-solubilized bullfrog collagen (ABFC) and 1,4-butanediol diglycidyl ether (BDE).

FIG. 2 shows (a) extraction yield of novel method; (b) SDS-PAGE image; and (c) ATR-FTIR profile of ABFC and bovine collagen (BV) (*p<0.05)

FIG. 3 shows (a) ATR-FTIR profile; and (b) in vitro degradation profile (*p<0.05).

FIG. 4 shows (a) brightfield microscopic images of macrophages' morphology on ABFC-BDE and BDE-crosslinked acid-solubilized BV (ABVC-BDE) samples; (b) gene expression analysis for immunogenic cytokines (*p<0.05); and (c) a diagram of the activation of inflammatory response (scale bar for (a)=200 μm, and scale bar for (c)=200 μm).

FIG. 5 shows the proliferation of (a) keratinocytes (HaCaTs); and (b) fibroblasts on 2D ABFC-BDE and ABVC-BDE samples.

FIG. 6 depicts the (a) focal adhesion of keratinocytes on 2D ABFC-BDE and ABVC-BDE samples; and (b) keratinocyte cell mobility studies of 2D ABFC-BDE and ABVC-BDE samples.

FIG. 7 shows (a) digital images of 3D ABFC-BDE and ABVC-BDE scaffolds; (b) microscopic FESEM images of 3D ABFC-BDE and ABVC-BDE scaffolds; (c) proliferation of keratinocytes on 3D ABFC-BDE and ABVC-BDE samples; and (d) proliferation of fibroblasts on 3D ABFC-BDE and ABVC-BDE samples (scale bar for (a)=3 mm).

FIG. 8 depicts LIVE/DEAD staining of HaCaT after 5 days of culture (green and red fluorescence represent the live cells and dead cells respectively, scale bar=400 μm; *p<0.05).

FIG. 9 shows (a) microscopic images of in vitro cell migration over 24 h; (b) wound gap closure over 24 h; and (c) 3D scaffold cell migration study (scale bar for (a)=200 μm, and scale bar for (c)=100 μm, *p<0.05).

FIG. 10 depicts the effect of disintegrin ‘KGD’ on cell adhesion on ABFC-coated surface.

FIG. 11 shows (a) AFM images of the coated surfaces; and (b) fibre diameter of collagen-coated surfaces (*p<0.05).

FIG. 12 shows (a) gene expression analysis for ECM deposition by fibroblasts on 2D ABFC-BDE and ABVC-BDE samples; and (b) gene expression analysis for ECM degradation by fibroblasts on 2D ABFC-BDE and ABVC-BDE samples.

FIG. 13 depicts the in vivo wound closure over 7 days and the percentage of closure after 7 days (Scale bar=5 mm).

FIG. 14 depicts (a) 2D thin collagen coating; (b) collagen patch; (c) collagen sponge; and (d) collagen filling (Scale bar=500 nm, gridlines represent 1 cm).

FIG. 15 depicts the antibacterial studies of (a) ABFC-BDE with no silver nanoparticles (Ag/NPs); and (b) ABFC-BDE loaded with 7.5 μg/mL of Ag/NPs against S. aureus.

FIG. 16 depicts the effect of bullfrog skin-derived collagen on different stages of wound healing process.

FIG. 17 depicts the timeline for the different collagen extraction methods: (a) new collagen extraction method; and (b) traditional acid solubilization method.

### DETAILED DESCRIPTION

Thus, in a first aspect of the invention, there is provided one or both of a crosslinked polymer matrix formed from a non-mammalian collagen and a crosslinking agent and a crosslinked polymer matrix formed from a non-mammalian collagen that has undergone self-crosslinking, wherein the non-mammalian collagen is type I collagen.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As will be appreciated, the crosslinked polymer matrix may be formed from:

a non-mammalian collagen and a crosslinking agent;

a non-mammalian collagen that has undergone self-crosslinking; or

a combination of both.

Herein all of these possibilities may be referred to as the crosslinked polymer matrix and this term should, unless otherwise specified, be understood accordingly.

The term “crosslinking agent” when used herein refers to a compound that can form at least two covalent bonds with the collagen, whether by inherently having at least two functional groups that can crosslink to the collagen or through reaction with itself to generate at least two functional groups capable of crosslinking. Any suitable crosslinking agent may be used. However, as the materials discussed herein may be used in wound dressings or as an implant in the human or animal body, the crosslinking agent may be a pharmaceutically acceptable crosslinking agent. For example, the crosslinking agent may be selected from one or more of the group consisting of genipin and compounds comprising two or more (e.g. 2, 3, 4, 5, 6, or 7) crosslinkable functional groups selected from the group consisting of amino, carboxylic acid, ester, aldehyde and epoxide functional groups. For example, the crosslinking agent may be selected from compounds comprising two or more crosslinkable functional groups selected from the group consisting of aldehyde and epoxide functional groups. Particular crosslinking agents that may be mentioned herein may have two crosslinkable functional groups.

Examples of suitable crosslinking agents include, but are not limited to, glutaraldehyde, genipin, 1,4-butanediol diglycidyl ether (e.g. the crosslinking agent is 1,4-butanediol diglycidyl ether) and combinations thereof. Examples of suitable crosslinking agents with two crosslinkable functional groups include, but are not limited to, glutaraldehyde and 1,4-butanediol diglycidyl ether (e.g. the crosslinking agent is 1,4-butanediol diglycidyl ether).

When present, the crosslinking agent may be used in any suitable amount of the crosslinked polymer matrix. It will be understood that the crosslinking agent will be covalently bonded to collagen. For example, the (covalently bonded) crosslinking agent may form from 3 to 15 wt %, such as from 4 to 10 wt % of the crosslinked polymer matrix formed from a non-mammalian collagen and a crosslinking agent. For example, in particular embodiments of the invention that may be mentioned herein, the crosslinking agent may form about 5 wt % of the crosslinked polymer matrix formed from a non-mammalian collagen and a crosslinking agent.

As noted above, the crosslinked polymer matrix may have undergone self-crosslinking. This may be achieved through the use of individual chemical reactions performed by a skilled person to affect self-crosslinking or, more particularly, it may be achieved by the use of one or more enzymes to affect the crosslinking. For example, the self-crosslinking may be achieved by the use of a transglutaminase. It will be appreciated that a self-crosslinked polymer matrix may not substantially contain the chemicals/enzyme(s) used to affect the self-crossliking. This is because these substances may be washed away during the processing of the crosslinked polymer matrix.

The effects of the polymeric material disclosed herein may be enhanced (e.g. synergistically) by the inclusion of an antibacterial compound. Any suitable antibacterial compound may be used herein. For example, the antibacterial compound may be one or more of chitosan, silver nanoparticles and antibiotics. In particular examples that may be mentioned herein, the antibacterial compound may be silver nanoparticles.

In embodiments of the invention, the polymeric material may use any suitable non-mammalian collagen. Any suitable non-mammalian species may be used to generate the type I collagen used in the polymeric materials disclosed herein. Such species include vertebrates and invertebrates. More particularly, the suitable non-mammalian collagen may be derived from a non-mammalian vertebrate. More particularly, the non-mammalian collagen may be derived from bullfrog skin, optionally wherein the bullfrogs belong to the genus rana (e.g. the bullfrogs are of the species Rana catesbeiana).

It is noted that the use of bullfrog skins to produce type I collagen enables the capture of a resource that may have otherwise been wasted. In addition, it is also noted that the use of collagen derived from non-mammalian resources may reduce or eliminate risks associated with disease from mammalian sources (e.g. bovine spongiform encephalopathy (BSE), transmissible spongiform encephalopathy (TSE) and foot-and-mouth disease (FMD). Therefore, non-mammalian sources of collages (e.g. bullfrog collagen) can be regarded as a safer source of collagen as compared to those from mammalian species.

The polymeric material disclosed herein may be particularly useful for applications relating to wound healing. As such, the polymeric material may be provided as a film, a sponge, a patch or a filling material. As will be appreciated, collagen is a highly fibrous material that, when crosslinked, may function as a hydrogel, resulting in the ability to absorb significant amounts of water. Given this, the polymeric material described herein may be used as a filling material to plug a wound, as it can expand its size and shape to that of the wound. This function may be particularly notable when the polymeric material has been lyophilised. In certain embodiments that may be mentioned herein, the polymeric material may be provided as a sponge or a patch.

A film may be formed by allowing a solvent in a solution of the polymeric material to evaporate. The film may be formed by spreading a small amount of the solution over a flat-bottomed container (e.g. a petri dish). Similarly, a sponge may be formed by placing a similar solution into a high-walled container (e.g. a well in a 96-well plate) and subjecting the solution to lyophilisation, thereby generating a porous sponge-like material. A patch may be formed by compression of the sponge (e.g. by cold pressing using a Carver Model 3889 auto C hot press machine for 10 min at 147.1 kN). The filling material may be any of the forms listed above and/or it may be a film that has been lyophilised. As will be appreciated, the sponge, patch and filling material may be porous in nature.

As intimated above, the polymeric material may be useful in wound healing. Thus, in an aspect of the invention, there is provided a wound dressing comprising a polymeric material as described herein. A wound dressing may be formed, for example (which is non-limiting), by crosslinking of collagen overnight, following by pouring the collagen solution into a larger mold (for instance a 5×5 cm rectangular mold) prior to freeze-drying/lyophilization to produce a collagen dressing with a final dimension of 5×5×0.3 cm (without cold pressing). In a further aspect of the invention, there is also provided a method of wound healing comprising the step of providing a suitable amount of a polymeric material as described herein to a subject in need thereof. As will be appreciated, the polymeric material used in the method may be provided in the form of a wound dressing as described herein.

It has been surprisingly found that, compared to existing materials that have been produced mostly from bovine collagen, the polymeric materials disclosed herein have been shown to possess intrinsic properties that accelerate re-epithelisation and wound closure. Without wishing to be bound by theory, it is believed that the adhesion of cells to the surface of the polymeric material disclosed herein is less relative to a corresponding material made using a mammalian collagen (e.g. crosslinked bovine collagen). Since cell migration requires a fine balance between cell adhesion and cell motility, cell movement is expected to be restricted on surfaces that are highly adhesive, thereby reducing the re-epithelisation and wound closure. This difference may be governed by the nature of non-mammalian collagen compared to that of mammalian collagen, such as the relative abundance and presentation of the peptide sequence (KGD (Lys-Gly-Asp)), that reduces cell adhesion sites and as a result, improves cell motility. Said KGD sequence may, for example be found in collagen derived from bullfrog skins.

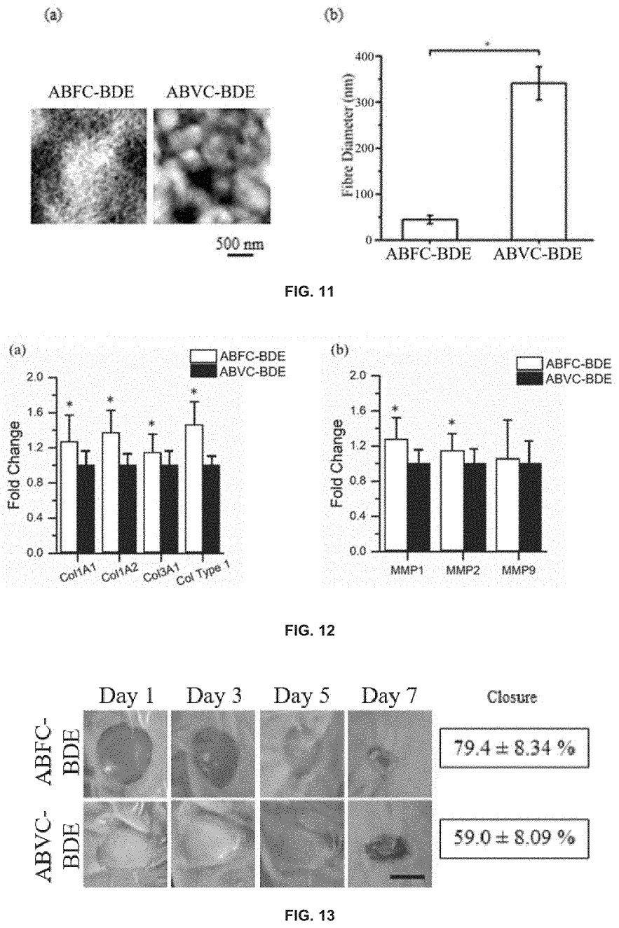

Without wishing to be bound by theory, it is believed that the improved re-epithelisation on the polymeric material compared to a corresponding mammalian-derived material may be due to the relationship between the fibre diameter of collagen and cell adhesion. The fibre diameter of the polymeric materials (i.e. bullfrog skin-derived collagen) disclosed herein is significantly smaller (approximately 20-25 nm in diameter and 200-400 nm in length) than that of mammalian-derived equivalents (micron-sized collagen fibrils) (FIG. 11(a)). Since equivalent mammalian-derived materials have a wider diameter, it is easier for the cells to adhere on each integrin binding sequence that is present at each D-period. In addition, the distance between the two integrin binding sequences may also be of high importance. This may be because it is believed that cellular adhesion is hindered by integrin-binding sites separated by more than ˜70 nm or less than ˜60 nm. The triple helical structure of bovine collagen has a 67 nm D-spacing and each D-period contains accessible integrin binding sequences. The bovine collagen used herein visibly exhibits a regular D-spacing of ˜67 nm in the collagen. Conversely, the lack of long range periodicity in the nano-bullfrog collagen fibers may result in moderate level of adhesion, thereby improving the re-epithelisation process compared to mammalian-derived materials

As mentioned hereinbefore, the collagen used in the polymeric materials above may be derived from a non-mammalian source. It has been surprisingly found that it is possible to generate substantial quantities of collagen through a revised method. Thus, in a further aspect of the invention, there is disclosed a method of providing a collagen precursor mixture from a non-mammalian source, the method comprising the steps of:

(a) providing a mixture of pre-treated skins from a non-mammalian animal in an acidic solvent; and
(b) subjecting the mixture to mechanical blending to provide the collagen precursor mixture in the form of a paste.

In particular, it has surprisingly been found that subjecting skins to mechanical blending enables not only the yield of collagen to be significantly increased compared to conventional methods, but also a substantial reduction in the processing time required. That is, compared to the commonly used acid solubilization methods for collagen extraction, this new method is much simpler and faster as it can be completed within approximately 11 days (FIG. 17a). Without wishing to be bound by theory, it is believed that the incorporation of the blending step, which cuts the skin into extremely small pieces, facilitates the isolation of collagen from the skin samples. The conventional method often involves repetitive and prolonged extraction steps to fully isolate the collagen from the skin tissue, which can take around 19 days for the collagen extraction (FIG. 17b). In addition, the extraction yield for the disclosed method may be around 70.0±7.5 wt % (based on 100 wt % of the skins), which is three times higher than the conventional method (21.3±3.6 wt %). Given the increase in collagen yield and reduction in time (much/all of which is spent at reduced temperatures (e.g. 4° C.)), the new process both reduces waste and costs.

In the method above, the acidic solvent may be aqueous acetic acid. The acid may have any suitable concentration in the aqueous media. In embodiments of the invention that may be mentioned herein, the aqueous acetic acid may have a molarity of from 0.1 to 1 M, such as from 0.3 to 0.7 M, such as about 0.5 M. The acidic solvent may be used in any suitable amount. For example, the acidic solvent may be provided in a weight to volume ratio of from 0.1:10 to 2:10, such as 1:10, where the weight refers to the weight of the pre-treated skins from a non-mammalian animal and the volume refers to the volume of the acidic solvent.

Any suitable form of blending may be used, provided that it shreds the skins into small pieces, such that a paste is formed. This blending may take place over any suitable time period. For example, the blending may be conducted over a period of from 1 to 20 minutes, such as from 2 to 10 minutes, such as about 5 minutes. The blending may make use of any suitable blender speed. For example, the blending may be conducted at from 20,000 to 50,000 rpm, such as from 30,000 to 40,000 rpm, such as about 35,000 rpm.

As the collagen within the skin of non-mammalian animals may be fragile, the method may be conducted at a temperature less than the ambient temperature of the environment. For example, the entire method may be conducted at a temperature of from 0.1 to 10° C., such as from 1 to 5° C., such as about 4° C.

While any suitable non-mammalian species may be used in the method disclosed herein, in certain embodiments that may be mentioned herein, the pre-treated skins may be bullfrog skin. For example, the bullfrogs may belong to the genus rana (e.g. the bullfrogs are of the species rana catesbeiana).

In order to transform the paste obtained from the method above, further downstream steps may be performed. Thus, there is also provided a method of providing collagen from a non-mammalian source, the method comprising the steps of:

(aa) providing a collagen precursor mixture in the form of a paste;
(ab) diluting the paste with water and centrifuging the resulting diluted paste to provide a collagen solution and a pellet comprising pigments and collecting the collagen solution;
(ac) adding an inorganic salt to the collagen solution for a period of time (e.g. from 12 to 48 hours, such as from 18 to 24 hours) to precipitate out a collagen salt, which is then collected by centrifugation;
(ad) adding an acidic solvent to the collected collagen salt to provide a free collagen mixture and subjecting the free collagen mixture to dialysis to provide a solution of collagen from a non-mammalian source.

As will be appreciated, this downstream method may be performed using the collagen precursor mixture in the form of a paste obtained from the method disclosed hereinbefore. This paste may be used as-is or, more particularly, it may be diluted. For example, the paste may be diluted by water in a ratio of from 1:2 to 1:10 vol/vol, such as from 1:3 to 1:7 vol/vol, such as about 1:5 vol/vol. Alternatively, the paste may be diluted by water in a ratio of from 1:10 to 1:30 vol/vol, such as from 1:10 to 1:20 vol/vol, such as about 1:10 vol/vol. As will be appreciated, the actual conditions may be selected by the skilled person depending on the amount of material and the apparatus used. For example, the dilution factor may be highly dependent on the volume/size of the centrifuge bottle. If the bottle has a large volume, then the particles may have to diffuse through a longer path before being deposited at the bottom of the bottle. As such, either a longer centrifuge time and/or more dilution is required when using a larger centrifuge bottle.

For the avoidance of doubt, it is explicitly contemplated that where a number of numerical ranges related to the same feature are cited herein, the end points for each range are intended to be combined in any order to provide further contemplated (and implicitly disclosed) ranges. Thus, above there is disclosed that the paste may be diluted by water in a ratio of:

from 1:2 to 1:3, from 1:2 to 1:5, from 1:2 to 1:7, from 1:2 to 1:10, from 1:2 to 1:20, from 1:2 to 1:30;
from 1:3 to 1:5, from 1:3 to 1:7, from 1:3 to 1:10, from 1:3 to 1:20, from 1:3 to 1:30;
from 1:5 to 1:7, from 1:5 to 1:10, from 1:5 to 1:20, from 1:5 to 1:30;
from 1:7 to 1:10, from 1:7 to 1:20, from 1:7 to 1:30;
from 1:10 to 1:20 vol/vol, from 1:10 to 1:30 vol/vol; or from 1:20 to 1:30 vol/vol.

The centrifugation step in step (ab) above may be conducted at from 15,000 to 50,000×g, such as from 20,000 to 35,000×g, such as about 25,000×g. This centrifugation step may be conducted for any suitable period of time. For example, the centrifugation may be conducted for a period of from 5 to 45 minutes, such as from 10 to 30 minutes, such as about 15 minutes.

Any suitable inorganic salt may be used in step (ac) of the method above. Suitable inorganic salts include, but are not limited to sodium sulphate, ammonium sulphate, alkali metal halides (e.g. potassium chloride, sodium chloride), and combinations thereof. In particular embodiments of the invention that may be mentioned herein, the salt may be sodium chloride. Any suitable concentration of the inorganic salt may be used. For example, the inorganic salt (e.g. sodium chloride) may be provided as an aqueous solution having a concentration of from 0.5 to 4.0 M, such as from 0.5 to 1.5 M, such as about 0.9 M.

The centrifugation step in step (ac) above may be conducted at from 3,000 to 10,000×g, such as from 4,000 to 6,000×g, such as about 5,500×g. This centrifugation step may be conducted for any suitable period of time. For example, the centrifugation may be conducted for a period of from 5 to 45 minutes, such as from 10 to 30 minutes, such as about 15 minutes. In certain embodiments, step (ac) of the method above may only be conducted once.

The acidic solvent in step (ad) above may be aqueous acetic acid. The acid may have any suitable concentration in the aqueous media. In embodiments of the invention that may be mentioned herein, the aqueous acetic acid may have a molarity of from 0.1 to 1 M, such as from 0.3 to 0.7 M, such as about 0.5 M.

The dialysis in step (ad) above may be conducted in two rounds, where:

(i) the first round dialysis may make use of aqueous acetic acid at a concentration of from 0.01 to 0.3 M, such as from 0.05 to 0.2 M, such as about 0.1 M; and

(ii) the second round dialysis may make use of water.

As the collagen within the skin of non-mammalian animals may be fragile, the method of generating collagen from a collagen precursor mixture in the form of a paste may be conducted at a temperature less than the ambient temperature of the environment. For example, the entire method may be conducted at a temperature of from 0.1 to 10° C., such as from 1 to 5° C., such as about 4° C.

The solution of collagen from a non-mammalian source obtained in the method above may be lyophilised.

Advantages that may be associated with the current invention include, but are not limited to the following.
A mechano-chemical method of collagen extraction from marine animal skins (e.g. bullfrog skin) that increases the yield up to 70% (w/w) with a 40% shorter extraction time.
Acid solubilised marine collagen (e.g. acid solubilised bullfrog collagen (ABFC)) may be extracted fully from food waste and successfully applied as a wound healing material by promoting keratinocytes (HaCaT) proliferation and re-epithelisation (HaCaT's motility) of the skin.
The unique nano-scale of the crosslinked collagen and/or presence of peptide sequence ‘KGD’ may act as a disintegrin which results in moderate cell adhesion favouring cell migration.
The polymeric materials disclosed herein may improve haemostasis and re-epithelisation—a crucial stage in tissue regeneration, by increasing cell migration with moderated cell adhesion.
The polymeric material can also be readily fabricated into various different forms such as thin film coating, patch, sponge or as filling material to play a role in different types of wounds. Hence, the polymeric material may be suitable for the development of future wound healing products.

Aspects and embodiments of the invention that may be mentioned herein include the following numbered statements.

1. A material comprising bullfrog skin-derived collagen and a cross-linker.

2. The material according to Statement 1, wherein the bullfrog is of the Rana catesbeiana species or are frogs belonging to the genus rang.

3. The material to Statement 1 or 2, wherein the cross-linker comprises an aldehyde and/or epoxy bifunctional cross-linker such as 1,4-butanediol diglycidyl ether (BDDE).

4. Use of the material according to any one of Statements 1 to 3 in wound healing.

Further aspects and embodiments of the invention will now be discussed by reference to the following non-limiting examples.

### EXAMPLES

Materials

Discarded American bullfrog (Rana catesbeiana) skins were kindly provided by KhaiSeng Trading & Fish Farm Pte Ltd. Sodium hydroxide (NaOH), acetic acid, sodium chloride (NaCl), sodium bicarbonate (NaHCO₃), 1,4-butanediol diglycidyl ether (BDE), paraformaldehyde (PFA), poly(hydroxyethyl methacrylate) (poly-HEMA), LIVE/DEAD® Cell Viability Assay (which consists of propidium iodide (PI) and fluorescein diacetate (FDA)), lipopolysaccharides (LPS), bovine serum albumin (BSA), KAPA SYBR® FAST qPCR Master Mix, Dulbecco's Modified Eagle's Medium/High Glucose (DMEM-HG) and Roswell Park Memorial Institute (RPMI)-1640 medium were purchased from Sigma-Aldrich, USA. THP-1 monocytes were purchased from ATCC, USA. Fetal bovine serum (FBS) was obtained from Research Instruments Pte Ltd, USA. Penicillin-Streptomycin, Trypsin-EDTA, goat anti-rabbit Alexa Fluor 488, Hoechst 33342, 10 K MWCO SnakeSkin™ dialysis tubing and PrestoBlue™ cell viability reagent were obtained from Thermo Fisher Scientific, USA. Bicinchoninic Acid (BCA) Protein Assay was purchased from Thermo Scientific—Pierce™, USA. Ethanol (EtOH), antihuman vinculin, and tetramethylrhodamine B isothiocyanate (TRITC)-conjugated phalloidin were purchased from Merck Millipore, USA. 10% polyacrylamide gel, iScript™ cDNA Synthesis Kit, T100™ Thermal Cycler, CFX Connect™ Real-Time PCR Detection System, Bio-Safe™ Coomassie Brilliant Blue R-250, Superscript® III First-Strand Synthesis Supermix, and Triton X-100 were purchased from Bio-Rad, USA. HaCaT cells were provided by Division of Genetics of Skin Carcinogenesis, Germany Cancer Research Center. Ibidi® culture inserts were purchased from Ibidi. Tegaderm™ was purchased from 3M. FSC 22 frozen section media was purchased from Leica Biosystems, USA. Phosphate buffered saline (PBS) was purchased from Gibco®. Type I collagenase solution was purchased from Life Technologies—Gibco®, NZ. RNeasy® Mini Kit was purchased from Qiagen, Germany. Difco™ Luria-Bertani broth and Difco™ Luria-Bertani agar were purchased from Miller, BD, USA.

Analytical Techniques

Sodium Dodecyl Sulphate-Polyacrylamide Gel Electrophoresis (SDS-PAGE)

The lyophilized collagen was dissolved in 0.01 M acetic acid at 10 mg/mL followed by mixing with an equal volume of 1×SDS Loading Dye and heated at 95° C. for 5 min to denature the protein. Subsequently, 10 μL of the solution was loaded onto the 10% polyacrylamide gel, where the proteins were separated under a current of 0.2 A for 2 h. Thereafter, the polyacrylamide gel was stained with Bio-Safe™ Coomassie Brilliant Blue R-250 for 1 h before washing with de-staining solution for another 1 h. Finally, the pictographs of Coomassie Brilliant Blue-stained polyacrylamide gel were captured to visualize the various protein bands.

Attenuated Total Reflection-Fourier Transform Infra-Red (ATR-FTIR) Spectroscopy

The samples were placed directly onto the ATR sampling accessory and scanned in a range of 4000-650 cm⁻¹ at a resolution of 4 cm⁻¹ using a Frontier MIR/FIR spectrometer (Perkin Elmer Inc., USA). The infrared spectrum was captured over 32 scans, where the peaks were used to identify the chemical functional groups. Further analysis was carried out on the obtained spectra to examine the structure of the extracted collagen by measuring the peak intensity ratio between Amide III and 1450 cm⁻¹ (J. K. Wang et al., Acta Biomater. 2017, 63, 246-260; and J. K. Wang et al., J. Mater. Sci.: Mater. Med. 2016, 27, 45).

Field Emission Scanning Electron Microscopy (FESEM)

Samples were sputtered with a thin layer of platinum at 20 mV for 15 s using a JEOL Auto Fine Coater (JFC-1600; JEOL Co., Japan). Following which, the samples were imaged with the JSM-7600F Schottky FESEM (JEOL Co., Japan) at an acceleration voltage of 5 kV at a magnification of ×200, ×15,000, and ×100,000.

Statistical Analysis

All experiments were carried out in triplicate (n=3) and expressed as mean±standard deviation unless otherwise specified. The statistically significant level was analyzed using Kruskal-Wallis non-parametric one-way analysis of variance and Mann-Whitney U test, where the data was considered statistically significant with p<0.05.

### Example 1

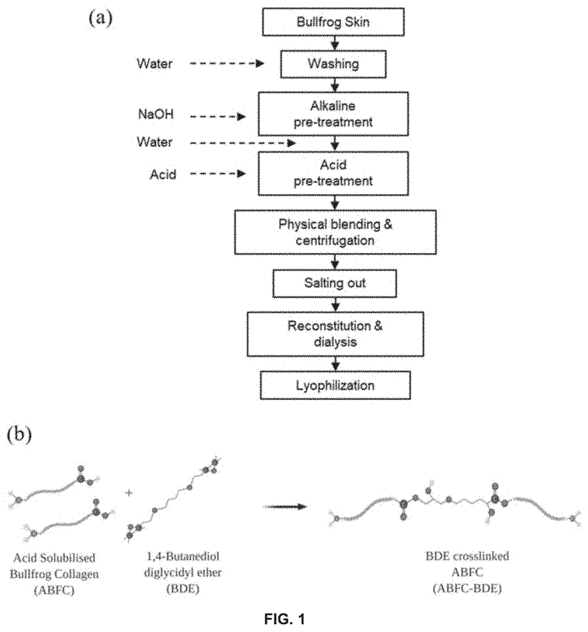

Acid-solubilized bullfrog collagen (ABFC) was extracted from American bullfrog (Rana catesbeiana) skins using a novel mechano-chemical method described below and in FIG. 1a.

All the extraction steps were performed at 4° C. and all the solutions involved were cooled to 4° C. prior to usage. Briefly, American bullfrog skins were washed with ice-cold distilled water to remove any blood and impurities. Afterward, the bullfrog skins were further cleaned with 0.5 M NaOH at a ratio (w/v) of 1:10 over 48 h with the solution changed at 24 h to remove the unwanted protein and black pigments. The bullfrog skins were then soaked in 0.5 M acetic acid at a ratio (w/v) of 1:10 for 24 h to release the collagen, followed by blending into a collagen paste using PHILIPS blender at 35,000 r.p.m. for 5 min. Subsequently, the collagen paste was further diluted with distilled water in 5 folds and centrifuged at 25,000×g for 15 min to remove the pigmentations. The clear collagen supernatant solution was incorporated with NaCl at a final concentration of 0.9 M to activate the salting of collagen for 24 h. At the end of the salt precipitation process, the collagen salt was collected by centrifugation process at 5,500×g for 15 min. Finally, the collagen salt was reconstituted in 0.5 M acetic acid to a point where the collagen salt was fully dissolved, to obtain a concentrated collagen solution, followed by dialysis using 10 K MWCO SnakeSkin® dialysis tubing against 0.1 M acetic acid and distilled water for 24 h each before lyophilization and storage at 4° C. until further usage.

### Example 2

To reinforce the stability and mechanical properties of collagen-based wound dressing materials, bi-functional epoxy-based cross-linker, BDE, was employed to crosslink acid-solubilised BV, and ABFC extracted in Example 1 to give ABVC-BDE and ABFC-BDE, respectively. The cross-linking mechanism is succinctly depicted in FIG. 1b, and the synthesis procedure of ABFC-BDE is provided below.

The lyophilized ABFC (10 mg/mL) was first dissolved in 0.5 M acetic acid. Subsequently, BDE was added to the ABFC solution at a concentration of 5% w/w BDE to collagen ratio and stirred at 300 rpm for 24 h at 4° C. At the end of the reaction, the reaction mixture (0.2 mL) was added into each individual well of a 24-well plate and air-dried to obtain 2D ABFC-BDE coat. EtOH treatment was used for the sterilisation of the 2D thin film by soaking the sample with 70% EtOH for 30 min, followed by washing with 1×PBS and cell culture medium. To prepare 3D ABFC-BDE porous patch, the reaction mixture (0.2 mL) formed after the crosslinking reaction was added into each individual well of a 96-well plate and lyophilised for one day. The 3D samples were sterilised using ethylene oxide (EtO) gas treatment using an EtOGas 4 steriliser (Andersen Products, Inc., USA) overnight.

### Example 3

The characterization of ABFC and 3D ABFC-BDE samples prepared in Examples 1 and 2, respectively, was carried out using SDS-PAGE and ATR-FTIR analyses, in vitro degradation test, and denaturation temperature of ABFC.

Denaturation Temperature

The extracted lyophilised collagen was soaked in 1×PBS overnight at 4° C., and 5-10 mg of the blotted dried collagen was placed in an aluminium hermetic pan. The test was carried out using a DSC-Q10 Analyser (TA Instruments, USA) with a heating rate of 2° C./min, and a temperature range of 10 to 100° C. under 50 mL/min nitrogen atmosphere. An empty pan was used as a reference. The endothermic peak temperature was measured as Td.

In Vitro Degradation Test

The collagen samples were immersed in 2 units/mL of Type I collagenase solution, and incubated at 37° C. for 28 days. The amount of collagen in the degraded filtrate was quantified daily using BCA Protein Assay Kit. The colourimetric changes were measured using a SpectraMax M2 microplate reader (Molecular Devices, USA) at 562 nm. Subsequently, the filtrate was replaced by fresh collagenase solution. A standard curve that associates known concentration of collagen with an absorbance value was used to convert the given absorbance value to collagen concentration.

Results and Discussion

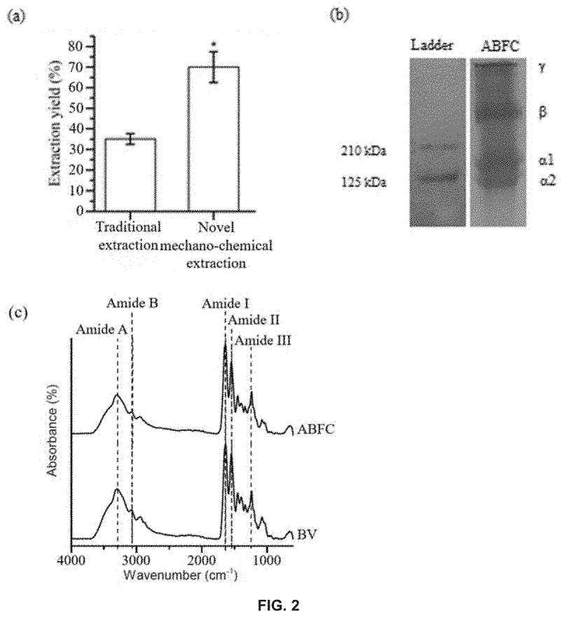

ABFC extracted via the novel mechano-chemical extraction method described in Example 1 gave a relatively high percentage yield of 70.0±7.5% (w/w), whereas the traditional extraction method only gave a percentage yield of 35.1±2.6% (w/w) (FIG. 2a). The type of ABFC was confirmed by SDS-PAGE analysis, in which the band width ratio of α1- and α2-chains was examined to be 2:1 (FIG. 2b), indicating that ABFC is a type I collagen. The comparison of the ATR-FTIR spectrum of ABFC with commercial type I collagen, BV, provided further support that ABFC is a type I collagen (FIG. 2c). The denaturation temperature of extracted ABFC was found to be 43.1±0.4° C., and is higher than both marine-sourced collagen and human body temperature, making ABFC a suitable scaffolding material for wound-healing applications.

TABLE 1
Amino acid composition of FSCOL (fish scale-derived
collagen), ABFC and BV (Wang, J. K. et al., J. Mater. Sci.: Mater. Med.
2016, 27, 45).

| Types of | FSCOL | ABFC | BV |
|---|---|---|---|
| amino acid | (residues/1000) | (residues/1000) | (residues/1000) |
| Aspartic acid | 41.40 | 49.29 | 48.45 |
| Threonine | 26.01 | 20.88 | 18.41 |
| Serine | 36.77 | 50.10 | 33.93 |
| Glutamic acid | 70.48 | 74.94 | 74.00 |
| Glycine | 383.70 | 393.94 | 367.72 |
| Alanine | 134.42 | 122.21 | 122.17 |
| Valine | 18.04 | 11.85 | 19.30 |
| Methionine | 5.11 | 0.00 | 0.00 |
| Isoleucine | 10.35 | 8.45 | 12.02 |
| Leucine | 20.41 | 20.90 | 30.68 |
| Phenylalanine | 11.36 | 10.70 | 12.12 |
| Lysine | [symbol] | 28.24 | 23.43 |
| Histidine | 7.10 | 6.43 | 5.20 |
| Arginine | 49.10 | 53.86 | 54.28 |
| Proline | [symbol] | 146.19 | 136.59 |

[symbol]

indicates data missing or illegible when filed

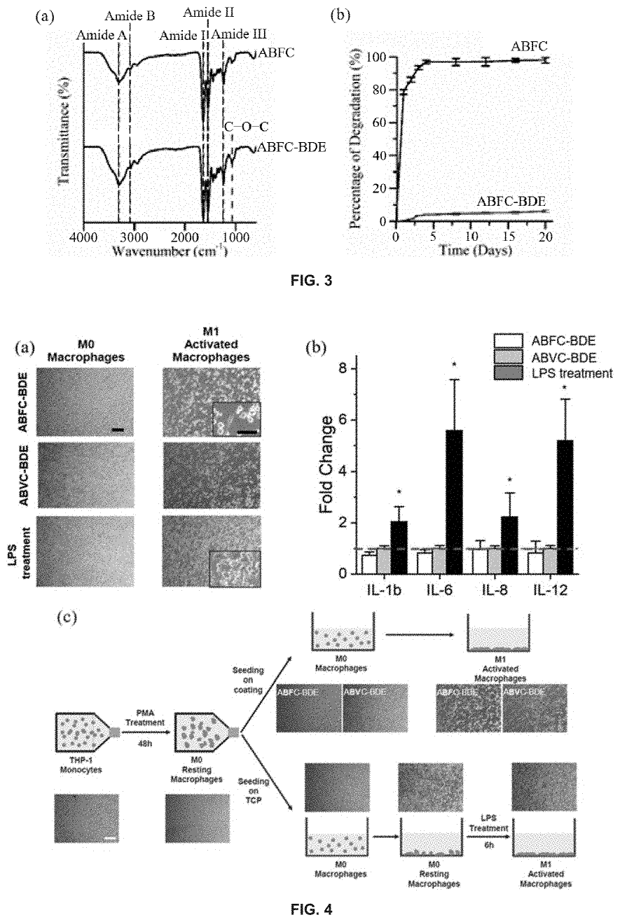

The successful crosslinking of ABFC with BDE was confirmed by ATR-FTIR spectroscopy. The accomplished crosslinking was confirmed by higher peak intensity at 1090 cm⁻¹ in ATR-FTIR analysis, which is an indication of ether linkages (C—O—C) produced during crosslinking (FIG. 3a). In addition, both ABFC and ABFC-BDE preserved their triple helical structures during the extraction and crosslinking processes as the peak ratio of transmittance at 1237 cm⁻¹ to 1450 cm⁻¹ was around 1.

In vitro degradation assay also proved that crosslinking improved degradation stability. Without crosslinking, ABFC degraded almost completely in 2 days, whereas crosslinked-ABFC-BDE retained its structural integrity over 20 days (FIG. 3b).

### Example 4

The immunogenicity of 2D ABFC-BDE and ABVC-BDE (prepared in Example 2) samples was evaluated as described below.

Cell Culture

THP-1 monocytes were cultured and expanded in RPMI-1640 medium supplemented with 10% FBS, 1.6 g/L sodium bicarbonate, and 1% Penicillin-Streptomycin under 37° C., 5% CO₂ environment, and saturated humidity.

Quantitative Polymerase Chain Reaction (q-PCR)

The macrophages were seeded on the collagen-coated surfaces, followed by inflammatory cytokine gene expression analysis via q-PCR. Trypsinised cells were collected, and the total mRNA was extracted using RNeasy® Mini Kit, according to the manufacturer's protocol. Thereafter, reverse transcription of extracted mRNA was performed using iScript™ cDNA Synthesis Kit, and the reaction was carried out in T100™ Thermal Cycler. Subsequently, the qPCR analysis was performed using KAPA SYBR® FAST qPCR Master Mix, and CFX Connect™ Real-Time PCR Detection System. Amplification of the template DNA was carried out for 40 cycles with 95° C. for 15 s for denaturation, and 60° C. for 30 s for amplification, and at the end of each cycle. For immunogenic response study, 1 μg/mL LPS was used as a positive control to induce polarization of resting macrophages (M0) to proinflammatory M1 phenotype.

Results and Discussion

The morphology of macrophages attached on ABFC-BDE was similar to that of ABVC-BDE but was reduced as compared to LPS treatment (FIG. 4a). In addition, gene expression of inflammatory cytokines on ABFC-BDE was comparable to ABVC-BDE, and was significantly lower than LPS-treated positive control (FIG. 4b). These results indicate that ABFC-BDE demonstrated a similar immunogenicity profile to ABVC-BDE and has a low immunogenicity.

### Example 5

The cytocompatibility of 2D and 3D ABFC-BDE and ABVC-BDE samples (prepared in Example 2) were compared by evaluating their effect on the proliferation and migration of human keratinocytes (HaCaT) and human dermal fibroblasts (HDF, ATCC. PCS201012™) as described below.

Cell Culture

HaCaTs and HDF were cultured in 75 cm² tissue culture flasks (T-75) with DMEM-HG supplemented with 10% FBS and 1× antibiotic-antimycotic at 37° C. and 5% CO₂ environment with saturated humidity. Cells were detached from the T-75 flask using 0.25% Trypsin-EDTA after cells reach 80% confluency to seed them onto 24-well plates for further cellular studies. Passages 4-13 for HaCaTs, and passages 4-10 for HDFs were used for these studies.

Cell Proliferation Protocol

The proliferation of HaCaT and fibroblast cells was determined using the PrestoBlue™ cell viability reagent at various pre-determined time points according to the manufacturer's recommended protocol. Briefly, the cells were seeded onto the 2D and 3D samples with a seeding density of 30 k cells/cm² and 600 k cells/mL, respectively. On pre-determined time points, the number of cells was measured by incubating the cell-seeded samples with 10% v/v PrestoBlue™ reagent for 1 h at 37° C. At the end of the incubation period, 200 μL of the solution was transferred into a 96-well plate where the fluorescence intensity (Ex 560/Em 590 nm) was measured using a SpectraMax M2 microplate reader (Molecular Devices, USA). The cell number was determined using a standard curve correlating the fluorescence intensity to the known number of cells. Further analysis was carried out to compare the population doubling rate of the HaCaT cells cultured on different samples according to Equation 1.

$$Cell doubling = \frac{ln ( {N}_{f} ) - ln ( {N}_{i} )}{ln ( 2 )} \qquad ( 1 )$$

Where N_{f} is the cell count obtained at the respective time point, and Nᵢ denotes the cell count acquired from the previous time point.

Focal Adhesion

HaCaTs (1×10⁴/cm²) were seeded onto 2D thin film samples, and incubated for 6 h at 37° C. and 5% CO₂ environment. After 6 h of incubation, nonadherent cells were gently washed away with 1×PBS, and adherent cells were fixed with 4% PFA for 15 min at room temperature. Subsequently, the cells were penetrated using 0.2% Triton X-100 for 10 min, and blocked using 2% BSA for 1 h at room temperature. For focal adhesion studies, the cells were treated with the primary antibody, antihuman vinculin (1:200 in blocking solution) overnight at 4° C., followed by secondary antibody treatment with goat anti-rabbit Alexa Fluor 488 (1:200 in blocking solution) with double labelling for actin cytoskeleton using TRITC-conjugated Phalloidin (1:200 in blocking solution) for 30 min at room temperature. The cells nuclei were stained using Hoechst 33342 (1:500 in 1×PBS) for 5 min prior to imaging with Zeiss Axio Observer Z1 inverted fluorescence microscope. The obtained images were analysed using an ImageJ freeware (https://imagej.nih.gov/ij/).

2D Cell Mobility

Silicon culture-inserts were sterilised with 70% EtOH, followed by washing with 1×PBS and cell culture medium. The sterilised inserts were then air-dried before placing onto the collagen-coated wells. HaCaTs cells (4×10⁴ cells/each chamber of silicon culture inserts) were seeded onto the collagen surface and incubated at 37° C. and 5% CO₂ environment with saturated humidity for 16 h. Subsequently, silicon culture inserts were removed, and the wells were washed with 1×PBS solution to remove the cell debris, and fresh serum-free medium was added for further incubation. Finally, the cell migration was imaged by Zeiss AxioVert light microscope (Carl Zeiss, Germany) at specific time points and analysed by ImageJ.

3D Cell Motility

48-well plate was coated with poly-HEMA (20 mg/mL in EtOH) to block the cell attachment to the plate. Subsequently, 3D porous constructs were placed into the individual wells of pHEMA-coated wells. A dense cell droplet (5×10⁵ cells/10 μL) was added onto the top surface of the 3D porous construct incubated at 37° C. and 5% CO₂ environment with saturated humidity for 2 h before topping up with fresh DMEM to cover the scaffolds fully. The cell-seeded scaffolds were collected at days 1, 3 and 5, and were fixed with 4% PFA for 1 h. The fixed samples were washed and soaked in FSC 22 Frozen Section Media prior to freezing at −20° C. and cryosectioned into 15 μm sections using a microtome (Leica Biosystems, USA). The sections were collected on poly-L-lysine treated glass slides (Thermo Fisher, USA), stained using Hoechst 33342 and imaged using Zeiss Axio Observer.Z1 inverted microscope. Images were analysed using an ImageJ freeware (https://imagej.nih.gov/ij/)

Results and Discussion

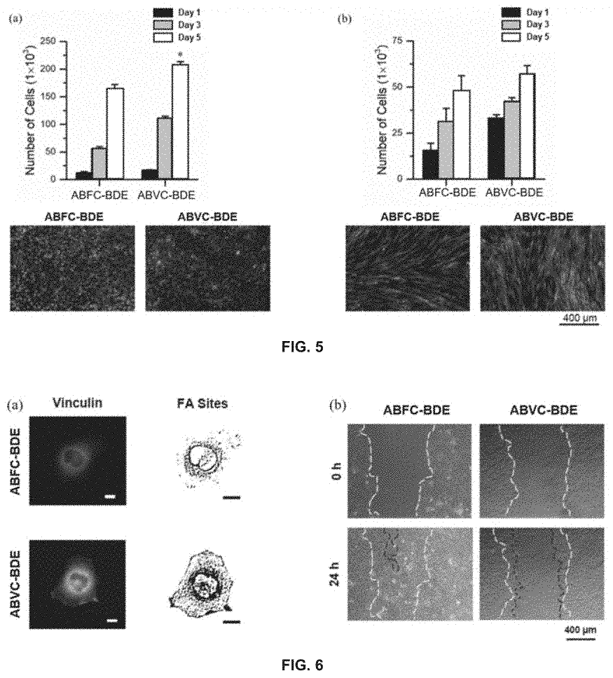

Both keratinocytes and fibroblasts seeded on 2D ABFC-BDE scaffold exhibited a positive proliferation profile alike to 2D ABVC-BDE scaffold (FIG. 5), indicating that ABFC-BDE is cytocompatible. Although ABFC-BDE promoted a smaller extent of proliferation than ABVC-BDE, ABFC-BDE enhanced keratinocytes migration more than ABVC-BDE. Focal adhesion studies (FIG. 6a) on 2D ABFC-BDE and ABVC-BDE samples showed that the number of focal adhesion sites on ABFC-BDE was 57% lower than on ABVC-BDE, and the area of focal adhesion sites on ABFC-BDE was 15% smaller than that on ABVC-BDE. In addition, keratinocyte cell mobility studies on 2D ABFC-BDE and ABVC-BDE samples demonstrated that ABFC-BDE displayed a 61% higher cell mobility rate than ABVC-BDE (FIG. 6b). As ABFC-BDE has the peptide sequence KGD (Lys-Gly-Asp), that reduces cell adhesion sites, it is possible that this feature improved cell motility as observed in the studies (Nykvist P. et al., J. Biol. Chem. 2001, 276, 38673-38679).

Similarly, 3D ABFC-BDE scaffold was porous and stable enough to support proliferation of keratinocytes and fibroblasts (FIG. 7)

### Example 6

The cytocompatibility of ABFC-BDE (prepared in Example 2) was further tested by using the protocol below to determine the cell-material interactions with HaCaT cells as cultured in Example 5.

Cell Viability Assay Protocol

The cell viability was also evaluated using a conventional LIVEDEAD assay on day 5. In general, FDA and PI were used to stain viable and dead cells, respectively (Boyd, V. et al., Curr. Trends Biotechnol. Pharm. 2008, 2, 66). Briefly, FDA (8 μg/mL) and PI (20 μg/mL) were mixed with serum-free DMEM, and the cells were treated with the staining solution for 5 min. Subsequently, cells were washed with 1×PBS and examined under Zeiss Axio Observer Z1 inverted fluorescence microscope (Carl Zeiss, Germany) fitted with a camera. FDA was observed with green fluorescence (488 nm; excitation wavelength and 515 nm; emission wavelength), and PI was observed with red fluorescence (543 nm; excitation wavelength and 615 nm; emission wavelength).

Results and Discussion

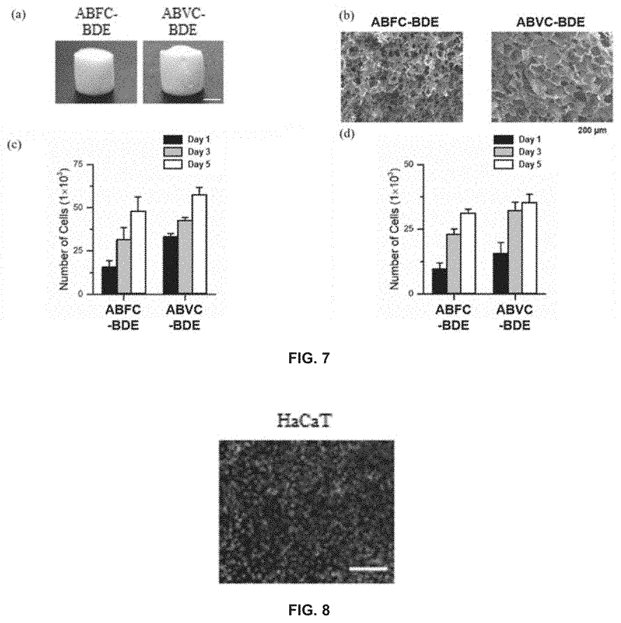

As shown in FIG. 6a, ABFC-BDE supported the attachment and proliferation of HaCaT cells over 5 days of culture. In addition, based on the LIVE/DEAD staining assay (FIG. 8), it was noted that the cells remained highly viable throughout the in vitro studies, suggesting that the ABFC-BDE is highly biocompatible.

### Example 7

The process of re-epithelisation during the healing stage is important to restore the barrier function of the skin and to minimize infection at the wound site. Re-epithelization occurs when epithelial cells from the wound edge migrate across the wound bed to form a new epithelium. In vitro cell migration assay is a useful tool to assess the re-epithelization process, and is indicative of keratinocytes functionality. Therefore, in vitro cell migration assay was employed to investigate the re-epithelisation capacity of 2D ABVC-BDE and ABFC-BDE, and 3D ABFC-BDE scaffolds (prepared in Example 2) by following the protocols in Example 5, where ABVC-BDE served as a control for further studies since BV has been used as a benchmark for wound healing applications.

Results and Discussion

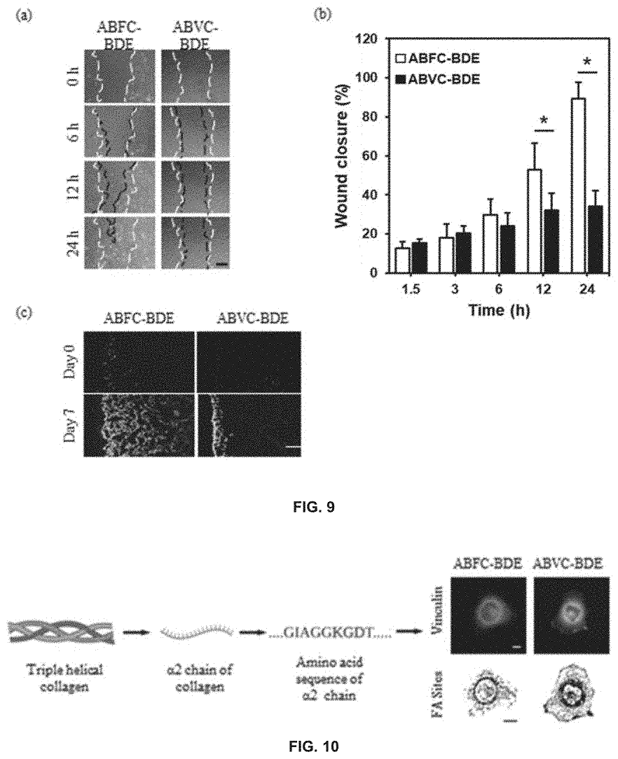

Remarkably, as shown in FIG. 9a-b, in vitro wound closure of HaCaTs was significantly faster on the 2D ABFC-BDE wound dressing material as compared to the 2D ABVC-BDE counterpart. Specifically, the in vitro wound was almost closed for the ABFC-BDE group whereas the closure was only 35% for the ABVC-BDE group after 24 h (FIG. 9a-b).

The fluorescence images of the porous 3D ABFC-BDE scaffolds are shown in FIG. 7b. HaCaT cells migration through the 3D scaffolds was observed over a course of 7 days. Consistent with the above 2D cell migration results, a higher penetration depth of the cells in the ABFC-BDE 3D sponge-like scaffold than in the ABVC-BDE group was observed (FIG. 9d).

### Example 8

To understand the enhanced re-epithelisation observed for ABFC-BDE, both 2D ABFC-BDE and ABVC-BDE scaffolds (prepared in Example 2) were taken for focal adhesion studies (described in Example 5) and fibre diameter measurement described below.

Fibre Diameter Measurement

ABFC-BDE and ABVC-BDE collagen were reconstituted in 0.5 N acetic acid at 100 μg/mL, then coated onto a mica disc. Briefly, 100 μL of the solution was added onto the mica disc for 1 min to allow physical absorption onto the surface, followed by excessive rinsing with deionised water and drying by nitrogen flow. AFM imaging was performed using a Cypher S (Asylum Research, Oxford Instrument, USA) with AC mode in the air with the ARC2 SPM controller and cantilever with imaging speeds of 1 Hz, scanning 256 lines. The samples were scanned with a surface area of 2 μm×2 μm. Finally, the obtained images were further processed to measure the fibre thickness and length of the collagen using an ImageJ freeware (https://imagej.nih.gov/ij/).

Results and Discussion

The enhanced re-epithelisation observed for ABFC-BDE could be attributed to cell adhesion. Compared to cells cultured on the ABVC-BDE substrate, the focal adhesion (adhesion proteins) of the human keratinocytes cultured on the ABFC-BDE is significantly smaller (FIG. 10). This suggests that the adhesion of the HaCaT cells is augmented on the ABVC-BDE surface, whereas the adhesion of the HaCaT cells on the ABFC-BDE surface is moderated. Since cell migration requires a fine balance between cell adhesion and cell motility, cell movement is therefore expected to be restricted on surfaces that are highly adhesive. From the materials standpoint, a possible molecular explanation could be attributed to the differences in peptide expression of collagen. As mentioned in Example 5, presence of non-adhesive peptide sequence such as, KGD (Lys-Gly-Asp), can potentially reduce cell adhesion sites and as a result, may contribute to improved HaCaT motility (Nykvist P. et al., J. Biol. Chem. 2001, 276, 38673-38679).

Another interesting observation is the stark differences in collagen fibre diameter of ABFC-BDE relative to ABVC-BDE. (FIG. 11) In the case of ABVC-BDE, the triple helical structure of bovine collagen has 67 nm D-spacing and each D-period contains accessible integrin binding sequences (Zeltz, C. et al., Biochim. Biophys. Acta 2014, 1840, 2533-2548). Since ABVC-BDE has a wider diameter, it is easier for the cells to adhere on the integrin-binding sequences.

### Example 9

The remodelling of extracellular matrix (ECM) in fibroblasts by 2D ABFC-BDE and ABVC-BDE samples (prepared in Example 2) was investigated and compared by measuring the gene expression of ECM secretion and remodelling genes by q-PCR analysis. The fibroblasts were cultured as described in Example 5, and q-PCR analysis was performed as described in Example 4.

Results and Discussion

As seen in FIG. 12a, ABFC-BDE enhanced ECM secretion more than ABVC-BDE. In addition, it was found that ABFC-BDE improved ECM remodelling via high expression of matrix metalloproteinases (MMPs) (FIG. 12b). Taken together, ABFC-BDE promoted better ECM remodelling than ABVC-BDE with balanced ECM deposition and ECM degradation.

### Example 10

The wound healing properties of ABFC-BDE were further evaluated in an in vivo study described below.

Two 8-mm diameter circular wounds were made with tissue punch on dorsal skin of C57BL/6 male mouse and 3D ABFC-BDE and ABVC-BDE scaffolds (prepared in Example 2) were placed on the wounds. Then, the wounds were covered with Tegaderm™ and wound closure was observed for 7 days.

Results and Discussion

Remarkably, closure of an 8 mm diameter full thickness wound over 7 days was significantly faster in the ABFC-BDE group than in the ABVC-BDE group by ˜20% (FIG. 13).

### Example 11

To treat different types of wounds, various forms of ABFC-BDE (prepared in Example 2) were fabricated as described below.

Fabrication of the Different Forms of ABFC-BDE

Thin Film Coating

To prepare a thin film coating, ABFC-BDE solution was cast onto the desired substrate, followed by air-drying.

Collagen Sponge

To fabricate a collagen sponge, ABFC-BDE solution was aliquoted into the desired mould, followed by freeze-drying to obtain the 3D porous collagen sponge.

Collagen Patch

ABFC-BDE collagen patch was fabricated by following the protocol for collagen sponge except with additional cold-pressing using a Carver Model 3889 auto C hot press machine for 10 min at 147.1 kN.

Collagen Filling Material

ABFC-BDE collagen filing material was fabricated by following the protocol for collagen sponge followed by mechanical disintegration to desired size.

Results and Discussion

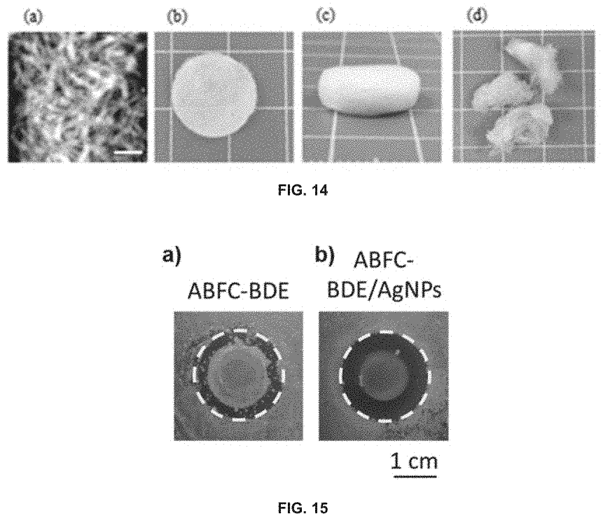

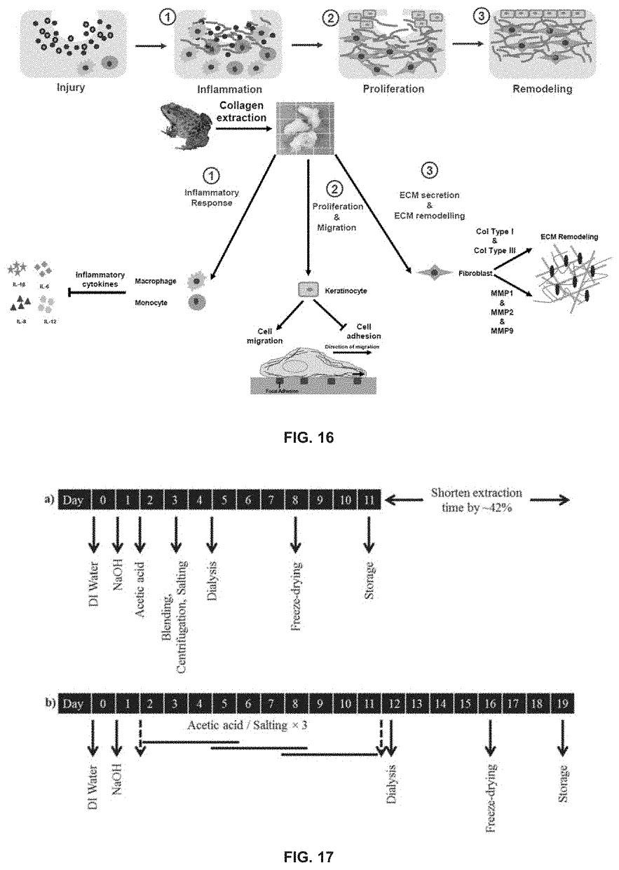

ABFC-BDE was fabricated into thin film coating, patch, sponge and filling material (FIG. 14) to play a role in different types of wounds. Hence, the extracted bullfrog skin-derived collagen in combination with our BDE cross-linking technology, could be a potential material for the development of future wound healing products.

### Example 12

Infection is one of the main challenges in the treatment of skin defects, in particular chronic wounds. As such, antimicrobial agents such as silver nanoparticles (AgNPs), antibiotics or any other bactericidal compounds can be incorporated into ABFC-BDE as a more effective therapeutic solution. 10 nm silver nanospheres (nanoComposix) was added to the ABFC-BDE precursor, and mixed overnight prior to lyophilization to produce ABFC-BDE/AgNPs. Thereafter, Staphylococcus aureus (S. aureus) ATCC 25923 were used to test the antibacterial potential of the ABFC-BDE/AgNPs. Antibacterial activity was determined by the Kirby-Bauer disc diffusion method described below.

Kirby-Bauer Disc Diffusion

The bacteria were grown overnight in Difco™ Luria-Bertani broth at 37° C. for 48 h in aerobic conditions. The disc diffusion method was performed on Difco™ Luria-Bertani agar, and the zone of inhibition was measured after 24 h of incubation.

Results and Discussion

After 48 h of incubation in S. aureus, a small zone of inhibition was observed in pristine ABFC-BDE group (12.4±3.6%) (FIG. 15a), which is attributed to the presence of residue acetic acid in the scaffold acting as a natural form of antiseptic agent in slowing the growth of S. aureus (Ryssel, H. et al., Burns 2009, 35, 695-700). However, a higher antibacterial activity was observed in the ABFC-BDE/NPs group in which a larger zone of inhibition against S. aureus was measured around corresponding silver nanoparticles-loaded ABFC-BDE (22.7±1.8%) (FIG. 15b). The presence of AgNPs was more potent in enhancing the antibacterial characteristic of the scaffold, and thus showing the potential of the developed ABFC-BDE as carrier material for the delivery of antibacterial agents.

What is claimed is:

1. A polymeric material comprising one or both of a crosslinked polymer matrix formed from a non-mammalian collagen and a crosslinking agent and a crosslinked polymer matrix formed from a non-mammalian collagen that has undergone self-crosslinking, wherein the non-mammalian collagen is type I collagen.

2. The polymeric material according to claim 1, wherein the crosslinking agent is a pharmaceutically acceptable crosslinking agent.

3. The polymeric material according to claim 1, wherein the crosslinking agent is selected from one or more of the group consisting of genipin and compounds comprising two or more crosslinkable functional groups selected from the group consisting of amino, carboxylic acid, ester, aldehyde and epoxide functional groups.

4. The polymeric material according to claim 1, wherein the crosslinking agent is selected from compounds comprising two crosslinkable functional groups.

5. The polymeric material according to claim 4, wherein the crosslinking agent is selected from one or more of the group consisting of glutaraldehyde and 1,4-butanediol diglycidyl ether.

6. The polymeric material according to claim 1, wherein, when present, the crosslinking agent forms from 3 to 15 wt % of the crosslinked polymer matrix formed from a non-mammalian collagen and a crosslinking agent.

7. The polymeric material according to claim 1, wherein when the crosslinked polymer matrix is formed from a non-mammalian collagen that has undergone self-crosslinking, the non-mammalian collagen has been crosslinked by a transglutaminase.

8. The polymeric material according to claim 1, wherein the polymeric material further comprises an antibacterial compound.

9. The polymeric material according to claim 8, wherein the antibacterial compound is selected from one or more of the group consisting of chitosan, silver nanoparticles and antibiotics.

10. The polymeric material according to claim 1, wherein the non-mammalian collagen is derived from bullfrog skin.

11. The polymeric material according to claim 1, wherein the polymeric material is provided as a film, a sponge, a patch or a filling material.

12. The polymeric material according to claim 11, wherein the polymeric material is provided as a sponge or a patch.

13. A wound dressing comprising a polymeric material as described in claim 1.

14. A method of wound healing comprising the step of providing a suitable amount of a polymeric material according to claim 1 to a subject in need thereof.

15. A method of wound healing comprising the step of providing a suitable amount of a wound dressing according to claim 13 to a subject in need thereof.

16. A method of providing a collagen precursor mixture from a non-mammalian source, the method comprising the steps of:
(a) providing a mixture of pre-treated skins from a non-mammalian animal in an acidic solvent; and
(b) subjecting the mixture to mechanical blending to provide the collagen precursor mixture in the form of a paste.

17. The method according to claim 16, wherein one or more of the following apply:
(ci) the acidic solvent is aqueous acetic acid;
(cii) the acidic solvent is provided in a weight to volume ratio of from 0.1:10 to 2:10, where the weight refers to the weight of the pre-treated skins from a non-mammalian animal and the volume refers to the volume of the acidic solvent;
(ciii) the blending is conducted over a period of from 1 to 20 minutes;
(civ) the blending is conducted at from 20,000 to 50,000 rpm;
(cv) the entire method is conducted at a temperature of from 0.1 to 10° C.; and
(cvi) the pre-treated skins are bullfrog skin.

18. A method of providing collagen from a non-mammalian source, the method comprising the steps of:
(aa) providing a collagen precursor mixture in the form of a paste;
(ab) diluting the paste with water and centrifuging the resulting diluted paste to provide a collagen solution and a pellet comprising pigments and collecting the collagen solution;
(ac) adding an inorganic salt to the collagen solution for a period of time to precipitate out a collagen salt, which is then collected by centrifugation;
(ad) adding an acidic solvent to the collected collagen salt to provide a free collagen mixture and subjecting the free collagen mixture to dialysis to provide a solution of collagen from a non-mammalian source.

19. The method according to claim 18, wherein one or more of the following apply:
(ba) the collagen precursor mixture is obtained using the method according to claim 16;
(bb) the paste is diluted by water in a ratio of from 1:2 to 1:10 vol/vol or the paste is diluted by water in a ratio of from 1:10 to 1:30 vol/vol;
(bc) centrifugation in step (ab) of claim 18 is conducted at from 15,000 to 50,000×g;
(bd) centrifugation in step (ab) of claim 18 is conducted for a period of from 5 to 45 minutes;
(be) inorganic salt in step (ac) of claim 18 is selected from one or more of sodium sulphate, ammonium sulphate, potassium chloride and sodium chloride;
(bf) centrifugation in step (ac) of claim 18 is conducted at from 3,000 to 10,000×g;
(bg) centrifugation in step (ac) of claim 18 is conducted for a period of from 5 to 45 minutes;
(bh) acidic solvent in step (ad) of claim 18 is aqueous acetic acid;
(bi) dialysis in step (ad) of claim 18 is conducted in two rounds, wherein:
(i) the first round dialysis makes use of aqueous acetic acid at a concentration of from 0.01 to 0.3 M; and
(ii) the second round dialysis makes use of water;
(bj) the entire method is conducted at a temperature of from 0.1 to 10° C.;
(bk) step (ac) of claim 17 is only conducted once.

20. The method according to claim 18, wherein the solution of collagen from a non-mammalian source is lyophilised.
